(19) United States
(12) Patent Application Publication
(10) Pub. No.: US 2010/0098745 A1
(43) Pub. Date: Apr. 22, 2010

(54) Methods for delivery of medication using dissolvable devices

(76) Inventor: Robert J. Staab, Old Lyme, CT (US)

Correspondence Address: Evelyn M. Sommer, Esq.;17th Floor, 570 Lexington Avenue, New York, NY, 10022-6894, US

(21) Appl. No.: 12/587,985

(22) Filed: Oct. 16, 2009

Related U.S. Application Data
(63) Continuation-in-part of application No. 12/287,253, filed on Oct. 8, 2008.
(60) Provisional application No. 60/998,383, filed on Oct. 11, 2007.

(51) Int. Cl. A61F 6/06; A61K 9/00

(52) U.S. Cl. 424/430; 424/422

(57) ABSTRACT

A method utilizing a dissolvable device for the internal delivery of medication and more particularly to the use of films, fibers or non-woven fabrics made of a safe polymer material incorporating a medication that is released by dissolution of the films, fibers or non-woven fabrics over time, and more particularly a treatment method for controlling or regulating the pH in the vagina by stabilizing and adjusting the pH in the vagina by minimizing the impact of the vaginal flora.

This application is a continuation-in-part of application Ser. No. 12/287,253 filed Oct. 8, 2008 which claimed the priority date of prior provisional application Ser. No. 60/998,383 filed on Oct. 11, 2007.

### FIELD OF THE INVENTION

The invention generally relates to dissolvable fibers and methods of making and utilizing dissolvable devices comprising fibers, non-woven fibrous structures including non-woven fabrics and masses of randomly intermingled fibers and films for the internal delivery of contraceptives and medication and more particularly to the use of films or fibers non-woven fabrics comprising fibers, made of pharmaceutically acceptable polymer materials incorporating a medication that is released by dissolution of the film, fibers or non-woven fabric made from such fibers over time. The present invention can be applied on or in a moist area of the body, such as the mouth, skin, vagina, rectum, ear canal, eye, etc. The present invention also relates to treatment methods for controlling or regulating the pH in the vagina by stabilizing and adjusting the pH in the vagina by delivery of an agent material incorporated in a dissolvable device minimizing the impact of the vaginal flora.

The method of using the invention comprises applying a device adapted for local administration of an agent material into a moist area of the body, such as the mouth, skin, vagina, rectum, ear canal and eye. The device comprises a dissolvable element and an agent material carried in such dissolvable element. During use, the heat and humidity in mouth, skin, vagina, rectum, ear canal or eye dissolves the dissolvable element and releases the agent. The dissolution properties of the dissolvable element can be readily tuned and controlled for rapid dissolution (for example in 5-60 seconds) or for dissolution over prolonged periods (for example up 12-24 hours), at least partially, by introducing nitrogen or other suitable gas in forming the film or fiber and therewith the non-woven fabric comprising the dissolvable element.

### BACKGROUND ART

Due to the growing awareness of medical complications associated with imbalance of vaginal microflora and/or its related effect on pH, it is often desirable to administer medication into the vagina and that the medication be applied throughout the area of the vaginal tract and cervix over an extended period of time, for example several hours or days. The remoter areas of the vaginal tract might not be readily reached by conventional vaginal suppositories due to the compact size and shape required for convenience of insertion. Also, because of the structure and shape of the vagina, inserted suppositories or tablets often do not stay in place, or upon melting, the medication may drain out of the vaginal passage thereby reducing the effectiveness of the applied medication. Medicated tampons also do not extend far enough or widely enough into the vaginal tract to deliver medication throughout the vaginal tract. Other types of rigid applicators have similar problems and are uncomfortable to insert and use. Thus, with the current methods, the desired medication may not be applied or maintained effectively in the vaginal tract for a sufficient period of time. This is true as well when the devices are for treating conditions in the oral cavity, wounds or infections of the skin and in underlying tissues, etc. More importantly, the known products suffer from deficiencies of the prior art products in that they do not dissolve readily and in addition are not stable on prolonged storage at high temperature and high humidity, such as is generally encountered in numerous tropical countries as well as seasonally in more temperate climates. Such products become, under exposure to adverse humidity conditions, sticky and excessively hygroscopic. To resolve this problem, prior art devices, employ expensive protective packaging such as foil packs which greatly increase the cost of the product to the end user. The high cost discourages their use particularly in areas of the world where the product is most needed. Moreover, foil packaging increases package components and since the foils used are not readily decomposable in landfills such packages have a disadvantageous environmental impact.

It should be noted that as used herein, high temperature means up to 140 degrees. F., high humidity means up to 99% relative humidity and prolonged storage means in excess of three years. The present inventor has previously been granted U.S. Pat. Nos. 5,529,782 and 5,393,528 on devices adapted for local administration of an agent material in an internal body cavity. The entireties of these patents are incorporated herein by reference thereto.

As used herein the term “non-woven” fibrous structure” is meant to include both the non-woven fabric and a mass of randomly intermingled fibers with or without compaction.

The term “non-woven fabric” as used herein denotes an assembly of fibers held together in a random web or mat by fusing or bonding the fibers.

It has occurred to the present inventor that the widespread condition of pH imbalance in the vagina and the changes in the microflora could be treated by application of a dissolvable element with adjustable dissolution properties which carries at least one kind of desired agent material into the vaginal cavity or other internal body cavity. The dissolvable element remains in substantially solid form before use and dissolves primarily due to human body temperature and moisture during use to release the agent material in a desired time release and dosage.

### SUMMARY OF THE INVENTION

In accordance with the present invention, there are provided methods of contraception and of treatment conditions the site of which is located in an internal body cavity. A preferred use is controlling and adjusting the pH of the vaginal flora content by utilizing devices made of a non-toxic polymer material incorporating a medication that is released by dissolution of the device, which can be a film, a fiber a non-woven fabric comprising fibers or a mass of randomly intermingled fibers with or without compaction. Suitable film devices have been disclosed in granted U.S. Pat. Nos. 5,529,782 and 5,393,528. In this invention, fibers and non-woven fabric devices prepared therefrom are disclosed wherein the fibers have incorporated therein a material agent or additive such as a contraceptive or medicinal agent, anesthetic, and the like. The fibers may also be packed or compressed together to form an inseparable mass much in the nature of a cotton ball. The compressed fiber masses are especially suitable for forming tampons and for use in topical applications as would be a cotton ball. Non-woven fabrics comprising fibers are prepared by spinning a mixture of an additive selected from the group of contraceptives, antimicrobials, anti-infective agents, pH modifying agents, therapeutic agents, skin softeners, cleaning agents and their mixtures and at least one dissolvable thermoplastic such as polyvinyl alcohol, polyethylene oxide and/or complex carbohydrate material. The fibers are then bonded together to form a composite non-woven fabric. Preferably the non-woven fabric is formed of at least one spunbonded layer of fibers formed from at least one dissolvable polymer and at least one additive. The non-woven composite structure may be in the form of a laminate in which the laminate consists of at least one fibrous layer made of the dissolvable polymer and additive and at least one further layer made of a thermoplastic polymer, such as a polypropylene spunbonded layer or a fibrous material such as a sheet of cellulose rayon or cotton card. The resulting fabrics can be formed into non-woven devices or structures in the form of vaginal tampons, contraceptive sponges, wound treatment gauzes and bandages. The non-woven fibrous structure can also comprise an assembly of fibers made of at least one dissolvable polymer and at least one additive held together by mechanical interlocking in a random manner. The resulting fibers with or without fusing or bonding can be used to make vaginal tampons, contraceptive sponges, packings, and treatment packings or applicators.

Each format of the device is adapted for local administration of an agent material in an internal body area such as the vagina and comprises a dissolvable element and an agent material carried in said dissolvable element wherein said dissolvable element is made of dissolvable polymer material, such as, polyvinyl alcohol, polyethylene oxide, and/or complex carbohydrate material, which is selected such that the dissolvable element remains in solid form before use, and dissolves due to human body temperature and moisture during use to release said agent material for local administration in the internal body area. The preferred dissolvable element is a film or a mass of the fibers held together by mechanical interlocking in a random manner or non-woven fabric formed from the fibers which have been made of polyvinyl alcohol, polyethylene oxide, and/or a complex carbohydrate material such as hydroxypropyl methyl cellulose which are safe, food-grade materials selected to obtain a desired release characteristic for the agent material. Two or more film layers may be combined as a laminate for compound release properties. The dissolvable element dissolves within the body area so that it does not have to be physically removed after use. It can also dissolve completely when flushed away, so that no plumbing blockage or ecologically disturbing solid waste occurs. In the case where the non-woven fibrous structure constitutes a mass of fibers, the fibers which form the structure may have the same composition and characteristics as in the case of a non-woven fabric.

The dissolution properties and texture of the dissolvable element may be modified by adding nitrogen or other suitable gases in forming the film or the non-woven fabric, as well as by the use of polyethylene oxide alone or in mixtures with polyvinyl alcohol and/or complex carbohydrate material. Forming the fibrous mass, film or non-woven fabric of the invention with different combinations of fibers and layers or polymer materials allows varied dissolution properties. The polyethylene oxide and complex carbohydrate materials add lubricity to the product as an added benefit. The composition of the dissolvable element is selected to have an improved heat and humidity stability, feel, texture, and dissolution time.

The active agent may be incorporated into either the entire portions of the device, i.e., as a homogeneous blend or in the case of a laminate, the device may include multiple layers, at least one layer containing one or more types of active materials. In the case of the fibrous structures, the fibers maybe prepared with different polymers and/or different active materials. The dissolvable element may also be used to deliver contraceptives or medications such as anti-infectives, anti-inflammatories, coronary vasodilators, anesthetics, antitussives, expectorants, estrogenic, progestational or prostaglandin agents, a homeopathic drug and the like, and combinations thereof. They may include fragrance, flavorants, coloring agents, preservatives, etc., to provide a more acceptable, environmentally sound product for consumers, and may also include a plasticizer or gas additive for better handling, lubricity, and/or release characteristics.

In the case of a film device, the film or the laminate formed of the films are preferably rolled into a cylindrically-shaped non-woven device. The cylindrically-shaped device can be digitally inserted into body cavity (vagina) during use as a digital tampon. In an alternative embodiment, the cylindrically-shaped non-woven device is encased in an applicator of the type conventionally used with tampons and the device can be delivered in the same manner as a tampon with an applicator.

In the case of a non-woven fibrous structure, both the fabric and the mass of fibers as provided it comprises at least one layer formed of fibers made of a dissolvable thermoplastic polymer and at least one beneficial additive. The non-woven structure is formed as a melt-extruded non-woven layer. Layers other than melt-extruded non-woven layers can be present. For example, one or more air-laid or wet laid tissue or other cellulosic fiber layers may be present to provide increased water absorbency. Alternatively, one or more layers of scrim or similar material may be present to add increased strength to the non-woven composite structure. The mass of fibers which comprise a physical combination of two or more distinct constituents, the polymers and active material combined at or prior to extrusion i.e.; produced in fiber form, the fibers being entangled i.e., randomly arranged, can be consolidated with heat and pressure or otherwise compacted to form a shaped device such as a tampon.

The term “melt-extruded” as applied to a non-woven layer is meant to include a non-woven layer or web prepared by any melt-extrusion process for forming a non-woven web in which melt-extrusion to form fibers is generally followed concurrently by web formation on a foraminous support. The term includes among others, such well-known processes as meltblowing, coforming, spunbonding, and the like. Such processes are exemplified by the following references:

(a) meltblowing references include, by way of example, U.S. Pat. No. 3,016,599 to R. W. Perry Jr., U.S. Pat. No. 3,704,198 to J. S, Prentice, U.S. Pat. No. 3,755,52 to 7 J. P. Keller et al., U.S. Pat. No. 3,849,241 to R. R. Butin et al., U.S. Pat. No. 39,185 to R. R. Butin et al. and U.S. Pat. No. 4,663,220 to T. J. Wisneski et al. See also V. A. Wente, “Superfine Thermoplastic Fibers, Industrial and Engineering Chemistry Vol. 48, No. 8, pp. 1342-1346 (1956); V. A. Wente et al., “Manufacture of Superfine Organic Fibers”, Navy Research Laboratory, Washington D.C., NRL Report4 364(III43I), dated May 25, 1954, United States Department of Commerce, office of Technical Services and Robert R. Butin and Dwight T. Lohkamp, “Melt Blowing—A One-Step Web Process for New non-woven Products”, Journal of the Technical Association of the Pulp and Paper Industry, Vol. 56, No. 4 pp. 74-77 (1973);
(b) coforming references (i.e., references disclosing a meltblowing process in which fibers or particles are comingled with the meltblown fibers as they are formed) include U.S. Pat. No. 4,1A0324 to R. A. Anderson et al. and U.S. Pat. No. 4,118,531 to E. R. Hauser; and
(c) spunbonding references include, among others, U.S. Pat. No. 3,341,394 to Kinney, U.S. Pat. No. 3,655,862 to Dorschner et al., U.S. Pat. No. 3,692,817 to Matsuki et al., U.S. Pat. No. 3,853,651 to Porte, U.S. Pat. No. 4,064,605 to Akiyama et al., 4,A91,140 to Harmon, U.S. Pat. No. 4,100,319 to Schwartz, U.S. Pat. No. 4,340,563 to Appel and Morman., U.S. Pat. No. 4,405,297 to Appel and Morman, U.S. Pat. No. 4,434,204 to Harmon et al., U.S. Pat. No. 4,627,811 to Gresiser and Wagner, and U.S. Pat. No. 4,644,045 to Fowells.

In general the required layer of dissolvable thermoplastic polymer is formed of any nontoxic material that is stable in solid form during storage, is compatible with the agent incorporated therein, and can be dissolved to release the agent during use due to the body's heat and moisture present.

### DESCRIPTION OF PREFERRED EMBODIMENTS

In the invention, the treatment for normalizing the vaginal flora by controlling or adjusting the pH of the vaginal cavity comprises inserting into the vaginal tract a dissolvable device for delivery of medication which device contains a dissolvable element made of a dissolvable polymer material, particularly, a mixture of polyvinyl alcohol, polyethylene oxide, and/or complex carbohydrate material, used for local administration of a pH adjusting or normalizing agent possibly with another medication agent, into an internal body area. The dissolvable polymer material is preferably a food-grade material safe for internal use. The dissolvable element is designed to be heat stable (e.g., up to 140 degree F.), and humidity stable (e.g., up to 99% relative humidity) so as to remain in substantially solid form and not begin dissolving before its intended use. Lubricity is another desirable property for use in the vagina where sensitive tissues are likely to be encountered.

The preferred dissolvable element is in the form of a film, a fibrous mass or a non-woven fabric made of the fibers formed from the combination of grades of polyvinyl alcohol, polyethylene oxide, and/or complex carbohydrate material. Polyvinyl alcohol (PVA) is a preferred material for the aforementioned devices because it is non-toxic and medically safe to use internally. PVA comes in different grades that can be classified as cold water soluble dissolves from 40° F. to 212° F.), intermediate dissolving (110° F. to 212° F.) PVA is commercially available from companies such as Air Products Company, of Allentown, Pa. The cold water soluble and intermediate dissolving grades are the most useful for the desired moisture and heat dissolving properties for contraceptive grade purposes. A particularly preferred cold water soluble grade of PVA is an 80% hydrolyzed polyvinyl alcohol having a molecular weight of 9,000-10,000; for intermediate solubility, an 87-89% hydrolyzed polyvinyl alcohol having a molecular weight of 13,000-23,000 for a slow dissolving, a 98-99% hydrolyzed polyvinyl alcohol having a molecular weight of 31,000 to 50,000 and for the least dissolving, a fully hydrolyzed >99% of polyvinyl alcohol having a molecular weight of 85,000-186,000 being preferred. All of the aforementioned polyvinyl alcohol preparations are available from Aldrich Chemical, Milwaukee, Wis. However, in the invention, a higher temperature or water soluble grade is preferred in order to alter the temperature dissolution and moisture, solubility and stability properties so that the film, mass of compacted fibers or non-woven fabric can be used most suitably in the internal body cavity environment. The PVA material or materials are selected to dissolve relatively quickly, e.g., over several minutes, or in some cases as little as several seconds but may be selected for a longer release time, such as several days.

Polyethylene oxide is another good material for the film, the fiber or non-woven fabric made of fibers because it has very good moisture, particularly humidity, stability and further is a food contact grade material. It is very compatible with the pH modifying agents such as ascorbic acid and lactic acid and many other medications. It also has the added benefit of good lubricity, which makes the device even more comfortable to insert and use. Preferred polyethylene oxide materials are sold by Aldrich Chemical of Milwaukee, Wis., in molecular weights of from 50,000 to 8,000,000 Daltons.

The use of inert gases such as nitrogen, in forming the film, the fiber or non-woven fabric to modify the dissolution properties of the dissolvable element formed from polyethylene oxide has been found to be equally favorable as their use in connection with the films, fibers or non-woven fabrics using polyvinyl alcohol as the dissolvable. The dissolution of the device can be readily adjusted by using different viscosities of the hydroxypropyl methyl cellulose ranging from less than 80 to more than 4,000 centipoises.

A complex carbohydrate material suitable for use in the film, fiber or non-woven fabric is hydroxypropyl methyl cellulose, or carboxy methyl cellulose which is sold, for example, under the trademark “Methocel” by Dow Chemical of Midland, Mich. This material is also food-grade, medically safe to use internally, low-cost, and very stable in a humid environment “Methocel” is cellulosic in nature being derived from trees. It is dissolvable in the same temperature ranges as PVA. Hydoxypropyl methyl cellulose is a particularly preferred material in the same temperature ranges as PVA. Its acceptance by the FDA as a direct food additive is well known (CAS 9004-65-3). The preferred hydroxypropyl methyl cellulose has an average molecular weight of about 86,000.

The dissolvable element may be a laminate of two or more layers of different polymer materials, or may be a single layer with two or more polymer ingredients mixed together. Further, the laminate may be a gas foamed layer or constructed of layers of both non-foamed and gas foamed polymer materials. The exact mixture used will depend upon the intended use and combination of properties and/or effects desired, which may include heat dissolving temperature range, time release period, lubricity, shelf life, turgidify, stability in a moisture environment, compatibility with spermicides and/or other medications etc. In the case of contraception, two layers may be used which dissolve at varying rates. Such a laminate device can offer prompt efficacy upon insertion combined with extended contraceptive protection with dissolution taking place over a period of many hours. Thus in accordance with the invention the device may be constructed as a laminate composed of gas foamed layer with a non-gas foamed polyvinyl alcohol layer and hydroxylpropyl methyl cellulose layer in all possible combinations. The laminates can be formed in the conventional manner.

Fully formed films or non-woven fabrics can also be laminated to each other through use of an adhesive. A preferred adhesive is a dilute aqueous solution of the polymer from which the film or non-woven fabric has been made. Thus, for example, a polyvinyl alcohol film could be adhered to another polyvinyl alcohol film through use of a dilute solution of polyvinyl alcohol.

The non-woven fabric or assembly of fibers are held together by fusing of the fibers or bonding with a cementing medium such as one of the resins used in making the fibers. The fibers may be oriented in one direction or may be deposited in a random manner.

The agent material to be administered locally in particular the agent for stabilizing and in adjusting the pH in the vagina, may include drugs, contraceptives or medications. The agent material is evenly distributed throughout the dissolvable device, so that as the dissolvable device slowly dissolves, it releases the agent material in the proper dosage to perform its pH adjusting effect and other medicating function. If a laminate of films or non-woven fabric is employed, more than one layer of the laminate can contain actives of the same or different identities. The agent material is selected for compatibility with the polymer material and its dissolution characteristics. The device of the invention thus is composed of a biologically compatible material that has been blended homogeneously with a pH adjusting or controlling agent possibly a spermicide and/or drug which is released into a body cavity at a controlled rate upon contact with the body fluid.

The dissolvable element may be used to deliver a single agent and/or medication, for example, the pH control or the adjustment agent alone, internally in the vagina or cervical area or in combination with other suitable medications which can be delivered with the film, or non-woven fabric made of fibers or mass of compacted fibers and include: (1) anti-infectives such as antibiotics, sulfonamides, antivirals, antifungals, antiprotozoan and antibacterials; (2) anti-inflammatories, such as hydrocortisone, dexamethasone, triamcinolone, and various prednisolone compounds; (3) estrogenic steroids, such as estrone; (4) progestational agents, such as progesterone; (5) prostaglandins; (6) coronary vasodilators; (7) antitussives; (8) antihistamines; (9) anesthetics, and (10) homeopathic drugs. Monoclonal antibodies such as those useful against cell surface components or against pathogenic organisms such as the human-immuno-deficiency (HIV) family of viruses may be incorporated into the device of the present invention for ultimate intravaginal release. Combinations of the various drugs may be used as desired. Typically the range of drug additives may be in the amount of 0.0001% to about 50% by weight. The pH stabilizing and/or adjusting agents and other medications may be in a variety of chemical forms, such as uncharged molecules, molecular complexes, or non-irritating, pharmacologically acceptable salts.

Simple derivatives of such medications, such as ethers, amides, and the like, can also be used for desirable properties such as retention, release, and easy hydrolyzation by body pH, enzymes, etc. The amount of medication to be used varies depending upon the condition, the particular drug, the desired therapeutic or prophylactic effect, and required release times. Examples of pH adjusting agents include ascorbic acid, vitamin C and the like. Other drugs include clotrimazole, miconazole, tioconazole, benzalkonium chloride, nystatin, dermally active steroids, hormones, benzocaine, sulfas, biologically prepared actives, psychotropics, nitroglycerine, etc. If the drug can be applied on or in a moist area of the body, such as the mouth, skin, vagina, rectum, ear canal, eye, etc. then the device can be used to deliver the drug effectively with timed release of the proper dosage. This should be an ideal way for treating ulcers of the mucous membranes and of the skin for adjusting and/or stabilizing the pH of the vagina as well as for treating burn wounds.

The dissolvable element may also include a plasticizer material, such as water, glycols, glycerin and like materials in order to enhance lubricity and softness. While water is suitable as a plasticizer it is not useful in all cases, but this factor can be readily ascertained. A preferred plasticizer is glycerin USP, sold by Van Waters & Rogers, Inc., in either natural or synthetic form. Glycerine (glycerol) CAS 56-81-5 is particularly preferred.

The plasticizer may be added in any desired concentration, for example, from 0.1% to 35%, for better handling and lubricity. The softness and flexibility of the dissolvable layer, due to its thin layer structure without any rigid elements, and particularly when combined with plasticizer, ensures that the device may be used with complete comfort.

Various preservatives, antifungal agents, antibacterial agents, antiviral agents, antiprotozoal agents, and antioxidants may also be added if desired. Flavors, fragrances, and/or coloring agents may also be added. The polymer films or fabrics may be substantially transparent, or may be embossed with indicia or colored with opaquing agents. These additives may be present in any desired concentration, for example, from 0.001% to 50%. The concentrations of these additives will depend upon the condition to be treated, the agent's desired properties, the agent to be released, the potency, the desired dosage, dissolution times, etc.

In preparation, the polymer solids, water, or other solvent, medicinal, glycerine etc. are admixed in the proper concentrations and the mixture heated to the appropriate temperature for dissolution and formation of a uniform blend to take place. The heating can take place, for example, by submerging vessels containing the mixture in water or jacketed vessels held at constant temperature, for example 104° F.-140° F. The mixture can be either be cast directly or transferred into another water bath of cooler ingredients introduced with stirring. The application of heat is, however, not necessary, which is advantageous when pharmaceuticals or other agents to be added are heat sensitive.

The fibers for forming the fabric are formed by melt-extrusion, and preferably by spinning a heated mixture which is then used as a laminate with the same or a different polymer-additive fibers layer or other nonwoven polymer or thermoplastic polymer layers or alternatively spunbonded rayon webs or webs of air laid or wet laid or carded rayon fibers of stable length using conventional procedures taking care not to alter the characteristics of the polymer or additive. Characteristics can be altered by adding appropriate amount of gas, such as air, nitrogen, or other inert gases, which can produce a more acceptable texture and modify the dissolution rates accordingly.

For example, it has been found that the addition of nitrogen or other inert gas to a PVA mixture containing nonoxynol-9 halves the dissolution rate. The fine tuning of dissolution rates and delivery of agent material, by the addition of gases and by altering the grades or mixtures of polymer materials or layers, is an important aspect of the present invention.

On addition of the gas, preferably nitrogen, a web is formed of the final formulation including the gas. The resultant structure can be described as a foam with various sized air bubbles trapped in the matrix. There is a dual benefit in that not only can the size of the bubbles in the foam alter the dissolution rates and correct what is a serious flaw in standard polymer films, it also offers to the user a perceptible softness of the film which enables the delivery of many types of drugs to tender mucosal tissues. It has been observed that the formation of this web of the polymer/drug formulation and the gas must be made just prior to casting on the glass or steel plates. This offers precise control over the microbubbles and resultant control over the dissolution.

Without the described web formation, the quick release of drug was heretofore not possible. This frothy foam mixture or web can also be added to a mold to provide a formed device such as a barrier delivery system which completely dissolves upon use in a body cavity, e.g. the vagina.

The gases, for example, air, or nitrogen are introduced near the point of application of the liquid polymer material to the stainless steel casting sheet. The gases are added in a closed system by mixing with whipping blades or a motor driven homogenizer to homogenize the mixture of polymer, active material and gas to form a frothy foam.

When the structure is more than one layer, one of the layers can be made of slower dissolving polymer material for release of more drug or another drug material in combination, for example a spermicide and an anti-infective or anti-inflammatory medication. A third (and additional) layer(s) with additional drug can also be employed for sustained release of the drug. Laminates of foamed or combinations of foamed with non-foamed layers are used to readily control the dissolution rate of the device. Thus, a composite of desired release properties and agent materials is obtained.

An agent may be provided which causes the additive to migrate to the surfaces of the fibers as they are formed during the melt-extrusion process and contains at least one functional group which imparts to the surfaces of the fibers at least one characteristic which is different from the surface characteristics of fibers prepared from the thermoplastic polymer alone. Examples of desired surface characteristic include, among others, hydrophilicity or water-wettability, hydrophobicity, antistatic properties, and alcohol repellency.

Additives which impart wettability to the surfaces of nonwoven fibers, as a consequence of the preferential migration of the additive to the surfaces of the fibers as they are formed, typically are siloxane-containing compounds.

The following examples are given for illustrating the invention and are not to be considered as a limitation of the invention's scope.

### Examples for Film Delivery of Ascorbic Acid pH Controlling Agent

### Example 1

| | |
|---|---|
| Carboxypropyl methyl cellulose | 165 mg |
| Glycerine | 20 mg |
| Ascorbic acid | 45 mg |

### Example 2

| | |
|---|---|
| Carboxypropyl methyl cellulose | 165 mg |
| Ascorbic acid | 45 mg |

### Example 3

| | |
|---|---|
| Polyvinyl alcohol | 165 mg |
| Glycerine | 20 mg |
| Ascorbic acid | 45 mg |

### Example 4

### Film for Delivery of Surfactant Spermicide

### Example 5

| | |
|---|---|
| Carboxypropyl methyl cellulose | 105 mg |
| Glycerine | 18 mg |
| Nonoxynol - 9 | 100 mg |

### Example 6

| | |
|---|---|
| Carboxypropyl methyl cellulose | 105 mg |
| Nonoxynol - 9 | 100 mg |

### Example 7

| | |
|---|---|
| Polyvinyl alcohol | 105 mg |
| Glycerine | 18 mg |
| Nonoxynol - 9 | 100 mg |

### Example 8

| | |
|---|---|
| Polyvinyl alcohol | 105 mg |
| Nonoxynol - 9 | 100 mg |

### Examples of Fibers for Use in Non-Woven Fabrics and Masses of Intermingled Fibers

### Example 9

| | |
|---|---|
| Polyvinyl Alcohol | 2,350 mg. |
| Ascorbic Acid | 250 mg. |

This would provide sufficient fibers to make a 2.6 gram tampon.

### Example 10

| | |
|---|---|
| Polyvinyl Alcohol | 2,350 mg. |
| Clotrimazole | 600 mg. |

This would provide sufficient fibers to make a 3.0 tampon.

Non-woven fabrics were produced from an assembly of fibers having the compositions of Examples 9 and 10 held together by interlocking of the fibers in a random web or mat by fusing of the fibers or bonding with a cementing medium such as the polyvinyl alcohol or other synthetic resin used in the manufacture of the fiber. The fibers may be oriented in one or more directions or deposited in a random manner.

A second method of preparing the film, fiber or non-woven fabric involves the addition of the active by way of a final water bath. There are typically several baths during the manufacturing process. The final bath can contain one or more actives for addition to the film a non woven to be formed.

A third method of preparing the film, fiber or non-woven fabric involves the inclusion of one or more actives in polymeric pellets. The pellets of the polymers can act as polyvinyl alcohol). can be manufactured containing one or more actives. When the polymer pellets are used for the production of the film, the actives are carried along with the pellets.

The method of the invention utilizing the dissolvable film, fiber or non-woven fabric, for internal delivery of pH adjusting and/or stabilizing agent medication in accordance with the present invention, has a number of advantages over use of conventional contraceptives and tampon and sponge applicators. It is fully dissolvable and environmentally safe for disposal, and does not require removal, cleaning refitting or replacement. It is made of natural, food grade materials for safe internal use, and can be manufactured free of any irritant or toxic chemicals. Tests show that the film, fiber or non-woven fabric can be made to dissolve to deliver the agent in less than half the time required for other film devices. It is very convenient and not messy to apply and use, and will not stain clothing as with creams and gels. It causes no irritation and can be readily inserted by hand and does not require an applicator. The film, fiber or non-woven fabric can also be used at the same time to administer desired medications in internal areas of the body.

Numerous variations are of course possible in the light of principles and examples disclosed above. All such variations are intended to be included within the entire spirit and scope of invention, as defined in the following claims.

1. A method for the delivery of an agent material into an internal body cavity for the delivery of medicines, contraceptives and for pH controlling, which comprises introducing a device characterized by improved heat and humidity stability into the cavity, a said device comprising at least one dissolvable element formed from a member selected from the group consisting of polyvinyl alcohol, polyethylene oxide, hydroxypropylmethyl cellulose and mixtures thereof.

2. A method according to claim 1, wherein said device is a film.

3. A method according to claim 1 wherein said device is a non-woven fabric formed from fibers.

4. A method according to claim 1 wherein said device is a fibrous structure comprising a mass of randomly intermingled fibers.

5. A method according to claim 1 wherein said body cavity comprises a member selected from the mouth, vagina, rectum, ear canal and eye.

6. A method according to claim 2 wherein said film is formed of a laminate comprising of at least two film layers, each of said film layer characterized by different temperatures and dissolution properties.

7. A method according to claim 2 wherein said dissolvable film additionally contains a spermicide for contraceptive use.

8. A method according to claim 1 wherein said agent material is ascorbic acid for pH regulation.

9. A method according to claim 2 wherein the dissolution rate of said film is adjusted by introducing an inert gas into said film.

10. A method according to claim 2, wherein said dissolvable film contains a medication which is a member selected from the group consisting of anti-infectives, anti-inflammatories, estrogenic steroids, progestational agents, prostaglandins, coronary vasodialators, antitussives, antihistamines, and anesthetics.

11. A method according to claim 4 wherein said device is a non-woven fibrous structure selected from the group consisting of non-woven fabric and masses of randomly intermingled fibers.

12. A method according to claim 2, wherein said film is rolled into a cylindrical body.

13. A method according to claim 12, wherein said cylindrical body is encased in an applicator prior to use.

14. A method for topical delivery of an agent material to an external body area using a device characterized by improved heat and humidity stability of up to 140° F. and up to 99% respectively which comprises applying said device to said external body area, said device comprising at least one dissolvable film formed from a member selected from the group consisting off polyvinyl alcohol, polyethylene oxide, hydroxypropyl cellulose and mixtures thereof, and at least one agent selected from the group consisting of spermicides, pH regulating agents, anti-infectives, anti-inflammatories, estrogenic steroids, progestational agents, prosaglandins, coronary vasodialators, antitussives, antihistamines, anesthetics, homeopathic drugs, monoclonal antibodies and mixtures thereof incorporated in said at least one dissolvable film, at least one of said films having distributed throughout an inert gas, wherein in use said device dissolves at a temperature of up to 140° F. and up to 99% relative humidity in the presence of the moisture present at said body area or added to release said agent material from said body area, whereby on dissolution of film said agent material is delivered topically to said external body area.

15. A method for topical delivery of an agent material to an external body area characterized by improved heat and humidity stability of up to 140° F. and up to 99% respectively which comprises applying said device to said device to said external body area, said device comprising at least one dissolvable element formed from a member selected from the group consisting of polyvinyl alcohol, polyethylene oxide, hydroxypropylmethyl cellulose and mixtures thereof, and at least one agent selected from the group consisting of spermicides, pH regulating agents anti-infectives, anti-inflammatories, estrogenic steroids, progestational agents, prostaglandins, coronary vasodialators, antitussives, antihistamines, anesthetics, homeopathic drugs, monoclonal antibodies and mixtures thereof incorporated in said first group member, wherein in use said device dissolves at the temperature of said cavity in the presence of the moisture naturally present therein to release said agent material.

16. A method according to claim 15, wherein said device is a non-woven fabric formed from fibers.

17. A method according to claim 15 wherein said device is a fibrous structure comprising a mass of randomly intermingled fibers.

18. A Non-woven structure comprising a plurality of fibers formed from fiber of a water dissolvable polymer material and/or a complex carbohydrate material having selected dissolution properties and an additive selected from a group consisting of cleansing agents, therapeutic agents, cosmetic agents, and contraceptive agents, whereby the structure remains in substantially solid form before use and dissolves due to human body temperature and moisture during use to release the additive in a desired timed release and dosage.

19. A non-woven structure according to claim 18 wherein said fibers additionally contain an agent which causes said additive to migrate to the surface of the fibers as they are formed.

20. A non-woven structure according to claim 18 wherein said polymer is selected from the group consisting of polyvinyl alcohol, polyethylene oxide, complex carbohydrate materials and mixtures thereof.

21. A non-woven structure according to claim 18 wherein the additive is ascorbic acid.

22. A non woven structure according to claim 18 wherein said additive is at least one of a spermicide anti-infective, anti-inflammatory, estrogenic steroid, progestational agent, prostaglandin, coronary vasodilator, sensory modification agent, cosmetic cleaning agent, pH modifying agent and topical anesthetics.

23. A non-woven structure according to claim 18 wherein said structure is a laminate.

24. A non-woven structure according to claim 18, wherein said laminate includes a layer of at least one of polymer material, a sheet of cellulose, rayon or cotton card.

25. A non-woven structure according to claim 18 wherein said agent causing migration of the additive to the surface of the fibers is a siloxane.

26. A non-woven structure according to claim 18 wherein said fiber is wetspun.

27. A non-woven structure according to claim 18 wherein said fiber is dryspun.

28. A non-woven structure according to claim 18 wherein said dissolvable polymer is foamed as a means for increasing its dissolution rate.

29. A non-woven structure according to claim 18 wherein said device is a non-woven fibrous structure selected from the group consisting of non-woven fabrics and a mass of randomly intermingled fibers.
